(19) United States
(12) Patent Application Publication
(10) Pub. No.: US 2006/0281144 A1
(43) Pub. Date: Dec. 14, 2006

(54) Method for utilizing nonparaffinophilic microorganisms for producing specific waste degradation

(76) Inventors: Mitchell S. Felder, Hermitage, PA (US); Harry R. Diz, Erie, PA (US); Justin Felder, Hermitage, PA (US)

Correspondence Address: ECKERT SEAMANS CHERIN & MELLOTT, 600 GRANT STREET, 44TH FLOOR, PITTSBURGH, PA, 15219, US

(21) Appl. No.: 11/450,560

(22) Filed: Jun. 9, 2006

Related U.S. Application Data
(60) Provisional application No. 60/689,490, filed on Jun. 10, 2005.

(51) Int. Cl. C12Q 1/04

(52) U.S. Cl. 435/34

(57) ABSTRACT

The present invention provides a method identifying nonparaffinophilic microorganisms suitable for biodegradation or bioremediation that do not substantially produce methane. The method provides the growing of nonparaffinophilic microorganisms on substrates in a bioreactor, wherein the bioreactor provides an environment conducive to the metabolism of many nonparaffinophilic microorganisms while being restrictive to methanogens and methane production. The method may further include substrates coated with a gelatinous matrix, wherein the gelatinous matrix coating baits the nonparaffinophilic microorganisms.

### CROSS REFERENCE TO RELATED APPLICATIONS

The present application claims priority to U.S. Provisional Ser. No. 60/689,490 entitled Method for Producing Specific Waste Degrading Bacteria in a Bioreactor.

### FIELD OF THE INVENTION

The present invention relates generally to the concentrated isolation and growth of hydrogen generating nonparaffinophilic microorganism cultures. More particularly, this invention relates to a method for the sustained growth of hydrogen using substrates coated with a gelatinous matrix. The sustained production is provided by nonparaffinophilic microorganisms wherein such nonparaffinophilic microorganisms form biofilm on the coated substrates.

### BACKGROUND OF THE INVENTION

There is further need in environmental interests for new developments of biodegredation. Biodegradation refers to the degradation of sewages, effluents, toxic substances or other material organic material by microorganisms. The breakdown of toxic substances is also known as bioremediation. Biodegredation typically occurs in aerobic or microaerobic environments, and is generally the process of converting organic materials back into methane, hydrogen, CO₂ and/or H2O through microbial action. Biodegredation is useful in that it breaks down unwanted or unneeded organic substances into natural substances. However, a typical biodegredation product may result in the formation of methane. Methane production in a bioreactor will allow methanogenic bacteria to rapidly multiply and overwhelm the hydrogen producing bacteria in the unit.

Thus, producing non-methane gases from biological systems, through biodegradation or bioremediation, wherein the energy for the process is substantially provided by naturally occurring bacteria, is an optimal solution. Use of nonparaffinophilic microorganisms to break down waste substances is one such possibility. “Nonparaffinophilic microorganism” means any microorganism sustained by a carbon source other than paraffin. Examples of such nonparaffinophilic microorganisms include, but are not limited to, the following: Clostridium (butyricum, welchii, pasteurianum, beikerincki) Methylotrohps (Methylomonas albus, Methylosinus tricosporium) Rumen bacteria (Ruminococcus albus) Archaea (Pyrococcus furiosus), Acetomicrobacterium, Acetomicrobium, Bacteroides, Desulfovibrio, Eubacterium, Escherichia coli, Enterbacer aerogenes, Klebsiella oxiloca, Kl. Pneumoniaw, Aeromonas, Alcaligenes, Campylobacter, Escherichia, Enterobacter, Hafnia, Proteus, Salmonella, Serratia. Streptococcus, Alcaligenes eutrophus, Bacillus licheniformis, Rhodospirillum rubrum, Rhodopseudomonas acidophilla, Rh. Capsulate, Rh. Gelatinus, Rh. Sphaeroides, Oscillatoria limnetica, Anabaena cylindrical A variabilis and Cynechococccus cedrorum.

Several methods of determining the presence or absence of nonparaffinophilic microorganisms have been previously disclosed, for example, U.S. Pat. No. 5,854,013 to Ollar et al, wherein a carbon based gelatinous matrix is used to bait nonparaffinophilic microorganisms. However, there has been no use of these techniques in biodegradation or bioremediation processes, and to further reduce the levels of methane produced during biodegradation or bioremediation.

Thus, there continually remains a need for simple and effective methods of bioremediation and biodegradation that do not produce substantial levels of methane.

### SUMMARY OF THE INVENTION

This need, and others, is met by the present invention which provides a method for identifying microorganisms suitable for biodegradation or bioremediation of a waste material, wherein the biodegradation or bioremediation does not substantially produce methane.

It is an object of the invention to provide a method for identifying microorganisms suitable for biodegradation or bioremediation, including the steps of selecting a waste material, heating the waste material to an increased temperature, introducing the waste material into a bioreactor, forming microorganism-containing biofilm on one or a multiplicity of substrates, selecting nonparaffinophilic microorganisms from the biofilm as nonparaffinophilic microorganisms able to biodegrade the waste material, and isolating the nonparaffinophilic microorganisms.

It is a further object of the invention to provide a method for identifying microorganisms suitable for biodegradation or bioremediation, including the steps of selecting a waste material, heating the waste material to an increased temperature, introducing the waste material into a bioreactor, forming microorganism-containing biofilm on one or a multiplicity of substrates, wherein the substrates are coated with a gelatinous matrix for baiting nonparaffinophilic microorganisms, selecting nonparaffinophilic microorganisms from the biofilm as nonparaffinophilic microorganisms able to biodegrade the waste material, and isolating the nonparaffinophilic microorganisms.

It is a further object of the invention to provide a gelatinous matrix coating, the gelatinous matrix coating formed from agar and at least one carbon compound.

It is a further object of the invention to provide a carbon compound selected from the list consisting of glucose, fructose, glycerol, mannitol, asparagines, casein, adonitol, l-arabinose, cellobiose, dextrose, dulcitol, d-galactose, inositol, lactose, levulose, maltose, d-mannose, melibiose, raffinose, rhamnose, sucrose. salicin, d-sorbitol, d-xylose or combination thereof.

It is a further object of the invention to provide a method further including the step adding concentrated amounts of the isolated nonparaffinophilic microorganisms to additional waste material to biodegrade the waste material.

Is a further object of the invention to provide a method wherein the waste material is heated to a temperature of about 60 to 100° C.

Is a further object of the invention to provide a method wherein the pH of the waste material is adjusted between about 3.5-6.0 pH at any point of the method.

These and other objects of the present invention will become more readily apparent from the following detailed description and appended claims.

### BRIEF DESCRIPTION OF DRAWINGS

FIG. 1 is a plan view of the hydrogen production system.

FIG. 2 is a side view of one embodiment of the bioreactor.

FIG. 3 is a plan view the bioreactor.

FIG. 4 is a plan view of coated substrates.

FIG. 5 is a top plan view of a system layout in a housing unit.

### DETAILED DESCRIPTION OF PREFERRED EMBODIMENTS

As used herein, the term “microorganisms” include bacteria and substantially microscopic cellular organisms.

As used herein, the term “waste material” includes organic waste material having carbon-based compounds.

As used herein, the term “methanogens” refers to microorganisms that metabolize hydrogen in one or a series of reactions that produce methane as one of the end products.

As used herein, the term “nonparaffinophilic microorganism” means any microorganism sustained by a carbon source other than paraffin.

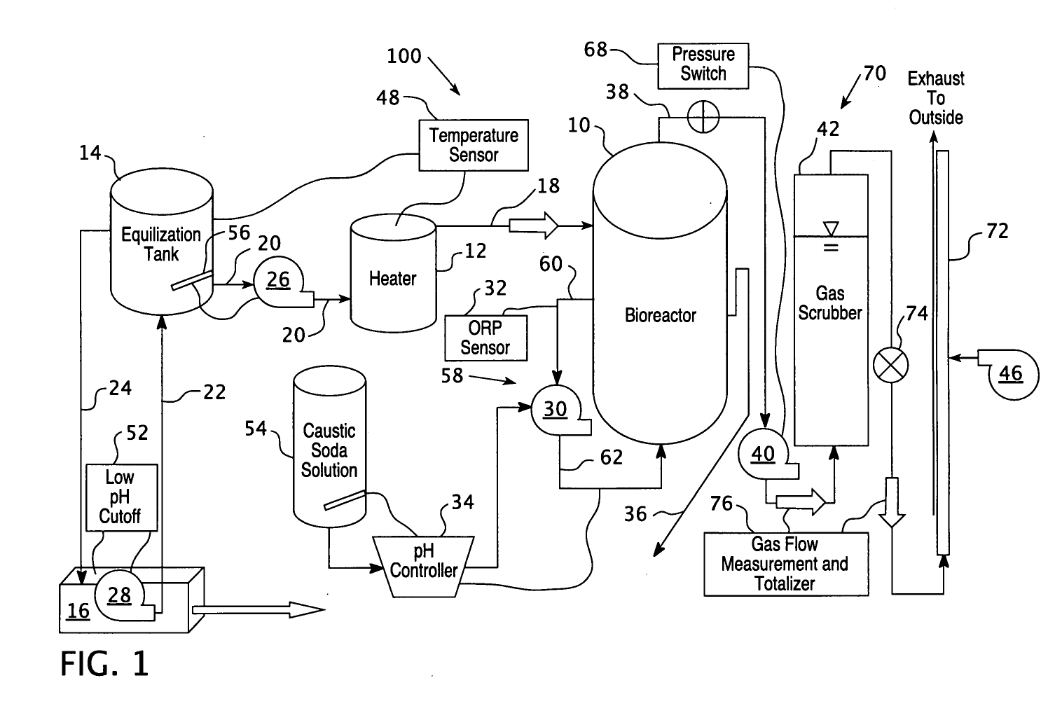

One embodiment of a method for sustained production of hydrogen in accordance with the present invention is shown in FIG. 1, wherein the method uses a system having bioreactor 10, heater 12, optional equalization tank 14 and reservoir 16. The method enables the baiting and growth of nonparaffinophilic microorganisms in bioreactor 10 that metabolize an organic waste material (hereinafter waste material) by using it as a feed, wherein the byproducts of the nonparaffinophilic microorganism metabolizing process do not substantially produce methane. Resultant gas byproducts that do not substantially include methane are produced by the biodegradation or bioremediation of a waste material by nonparaffinophilic microorganisms. The waste material may be any material that one wishes to degrade or detoxify, for example, sugar-containing waste waters, wastewaters rich in protein and fats, such as milk product wastes, and sewage-related wastes, such as municipal sewage. However, any organic feed material containing organic material is usable.

In one embodiment, nonparaffinophilic microorganisms metabolize sugars in the waste material under the reactions:
Glucose→2 Pyruvate (1)
2 Pyruvate+2 Coenzyme A→2 Acetyl-CoA+2 HCOOH (2)
2 HCOOH→2 H₂+2 CO₂ (3)

In this embodiment, one mole of glucose produces two moles of hydrogen gas and carbon dioxide. In alternate embodiments, other waste materials include agricultural residues and other organic wastes such as sewage and manures. Typical nonparaffinophilic microorganisms are adept at metabolizing the high sugar organic waste into bacterial waste products. The waste material may be further treated by aerating, diluting the waste material with water or other dilutants, adding compounds that can control the pH of the organic feed material or other treatment step. For example, the electrolyte contents (Na, K, Cl, Mg, Ca, etc.) of the waste material can be adjusted. Further, the waste material may be supplemented with phosphorus (NaH₂PO₄) or yeast extract.

Waste material provides a plentiful feeding ground for nonparaffinophilic microorganisms and is naturally infested with these microorganisms. In preferred embodiments, the nonparaffinophilic microorganisms are preferably microorganisms that thrive in pH levels of about 3.5 to 6.0 and can survive at elevated temperatures, thereby enabling metabolism under conditions unfavorable to methanogens.

In one embodiment of the invention, waste material is first contained in reservoir 16. Reservoir 16 is a container known in the art that can contain a waste material. The size, shape, and material of reservoir 16 can vary widely within the spirit of the invention. In one embodiment, reservoir 16 is one or a multiplicity of storage tanks that are adaptable to receive, hold and store the waste material when not in use, wherein the one or a multiplicity of storage tanks may be mobile. In other embodiments, reservoir 16 is a well that is adaptable to receive and contain wastewater and/or effluent directly from an industrial process. For example, reservoir 16 may be adaptable to receive and contain wastewater that is effluent from a juice manufacturing industrial process, such that the effluent held in the reservoir is sugar rich juice sludge.

If reservoir 16 is a well for capture of waste material directly from an industrial process, the method of the invention is preferably used in proximity with an industrial facility. The industrial facility emits waste products, such as organic rich effluent, which is thereafter captured by reservoir 16. By keeping proximity of the method to the industrial facility, the method provides a compact and cost effective method of biodegradation that acts on unwanted waste products of an industrial facility to produce substantially non-methane containing gas.

The waste material in reservoir 16 is thereafter conveyed throughout system 100, such that the system is preferably a closed system of continuous movement. Conveyance of waste material can be achieved by any conveying means known in the art, for example, through passages operably related to one or a multiplicity of pumps. The method preferably uses a closed system, such that a few well placed pumps can convey the waste material throughout the system, from reservoir 16 to optional equalization tank 14 to heater 12 to bioreactor 10 to outside of bioreactor 10. In preferred embodiments, waste material contained in reservoir 16 is conveyed into passage 22 with pump 28. Pump 28 is in operable relation to reservoir 16 such that it aids removal movement of waste material 16 into passage 22 at a desired, adjustable flow rate, wherein pump 28 can be any pump known in the art suitable for pumping liquids. In a preferred embodiment, pump 28 is a submersible sump pump.

In some embodiments, the method may further include temporary deactivation of conveyance from reservoir 16 to equalization tank 14 or heater 12 if the pH levels of waste material in reservoir 16 exceeds a predetermined level. In this embodiment, reservoir 16 furthers include a low pH cutoff device 52, such that exiting movement into passage 22 of the waste material is ceased if the pH level of the waste material is outside of a desired range. The pH cutoff device 52 is a device known in the art operably related to reservoir 16 and pump 28. It the monitor detects a pH level of a waste material in reservoir 16 out of range, the device ceases operation of pump 28. The pH cut off level in reservoir 16 is typically greater than a preferred pH of bioreactor 10. In preferred embodiments, the pH cutoff level is set between about 7 and 8 pH. The conveyance with pump 28 may resume when the pH level naturally adjusts through the addition of new waste material into reservoir 16 or by adjusting the pH through artificial means, such as those of pH controller 32. In alternate embodiments, particularly when reservoir 16 is not adapted to receive effluent from an industrial process, the pH cutoff device is not used.

Passage 22 provides further entry access into equalization tank 14 or heater 12. Equalization tank is an optional intermediary container for holding waste material between reservoir 16 and heater 12. Equalization tank 14 provides an intermediary container that can help control the flow rates of waste material into heater 12 by providing a slower flow rate into passage 20 than the flow rate of waste material into the equalization tank through passage 22. An equalization tank is most useful when reservoir 16 received effluent from an industrial facility such that it is difficult to control flow into reservoir 16. The equalization tank can be formed of any material suitable for holding and treating the waste material. In the present invention, equalization tank 14 is constructed of high density polyethylene materials. Other materials include, but are not limited to, metals or plastics. Additionally, the size and shape of equalization tank 14 can vary widely within the spirit of the invention depending on output desired and location limitations.

The method preferably further includes discontinuance of conveyance from equalization tank into heater 12 if the level of waste material in equalization tank 14 falls below a predetermined level. Low-level cut-off point device 56 ceases operation of pump 26 if waste material contained in equalization tank 14 falls below a predetermined level. This prevents air from being sucked by pump 26 into passage 20, thereby maintaining an anaerobic environment in bioreactor 10. Waste material can be removed through passage 20 or through passage 24. Passage 20 provides removal access from equalization tank 14 and entry access into heater 12. Passage 24 provides removal access from equalization tank 14 of waste material back to reservoir 16, thereby preventing excessive levels of waste material from filling equalization tank 14. Passage 24 provides a removal system for excess organic feed material that exceeds the cut-off point of equalization tank 14. Both passage 20 and passage 24 may further be operably related to pumps to facilitate movement of the waste material. In alternate embodiments, equalization tank 14 is not used and waste material moves directly from reservoir 16 to heater 12. This is a preferred embodiment when the method is not used in proximate conjunction with industrial facility such that effluent from the industrial facility is directly captured in reservoir 16. If reservoir 16 is one or a multiplicity of storage tanks holding a waste material, equalization tank 14 may not be necessary. In these embodiments, passages connecting reservoir 16 and heater 12 are arranged accordingly.

The waste material is heated prior to introduction into the bioreactor to deactivate or kill undesirable microorganisms, i.e., methanogens and non-hydrogen producers. The heating can occur anywhere upstream. In one embodiment, the heating is achieved in heater 12, wherein the waste material is heated within the heater. Alternatively, waste material can be heated at additional or alternate locations in the hydrogen production system. Passage 20 provides entry access to heater 12, wherein heater 12 is any apparatus known in the art that can contain and heat contents held within it. Passage 20 is preferably operably related to pump 26. Pump 26 aids the conveyance of waste material from equalization tank 14 or reservoir 16 into heater 12 through passage 20, wherein pump 26 is any pump known in the art suitable for this purpose. In preferred embodiments, pump 26 is an air driven pump for ideal safety reasons, specifically the interest of avoiding creating sparks that could possible ignite hydrogen. However, motorized pumps are also found to be safe and are likewise usable.

To allow nonparaffinophilic microorganisms within the bioreactor 10 to metabolize the waste material and produce gas not substantially containing methane, methanogens contained within the waste material are substantially killed or deactivated. In preferred embodiments, the methanogens are substantially killed or deactivated prior to entry into the bioreactor. In further preferred embodiments, methanogens contained within the waste material are substantially killed or deactivated by being heated under elevated temperatures in heater 12. Methanogens are substantially killed or deactivated by elevated temperatures. Methanogens are generally deactivated when heated to temperatures of about 60-75° C. for a period of at least 15 minutes. Additionally, methanogens are generally damaged or killed when heated to temperatures above about 90° C. for a period of at least 15 minutes. In contrast, many nonparaffinophilic microorganisms are resistant to temperatures up to about 110° C. for over three hours. Heater 12 enables heating of the waste material to temperature of about 60 to 100° C. in order to substantially deactivate or kill the methanogens while leaving non-methanogen nonparaffinophilic microorganisms substantially functional. This effectively pasteurizes or sterilizes the contents of the waste material from active methanogens while leaving the non-methanogen nonparaffinophilic microorganisms intact, thus allowing the produced biogas to include gas and not substantially methane. Heater 12 can be any receptacle known in the art for holding, receiving and conveying the waste material. Similar to the equalization tank 14, heater 12 is preferably formed substantially from metals, acrylics, other plastics or combinations thereof, yet the material can vary widely within the spirit of the invention to include other suitable materials. Similarly, the size find the shape of heater 12 can vary widely within the spirit of the invention depending on output required and location limitations. In preferred embodiments, retention time in heater 12 is it least 45 minute, preferably between 45 and 90 minutes. Retention time marks the average time any particular part of waste material is retained in heater 12.

To maintain temperatures at desired levels, at least one temperature sensor 48 monitors a temperature indicative of the waste material temperature during the heating step, preferably the temperature levels of equalization tank 14 and/or heater 12. In preferred embodiments, an electronic controller is provided having at least one microprocessor adapted to process signals from one or a plurality of devices providing waste material parameter information, wherein the electronic controller is operably related to the at least one actuatable terminal and is arranged to control the operation of and to controllably heat the heating tank and/or any contents therein. The electronic controller is located or coupled to heater 12 or equalization tank 14, or can alternatively be at a third or remote location. In alternate embodiments, the controller for controlling the temperature of heater 12 is not operably related to temperature sensor 48, and temperatures can be adjusted manually in response to temperature readings taken from temperature sensor 48.

Waste material is then conveyed from heater 12 to bioreactor 10. Passage 18 connects heater 12 with bioreactor 10. Waste material is conveyed into the bioreactor through transport passage 18 at a desired flow rate. When pumps are operating and not shut down by, for example, low pH cut off device 52, the system is preferably a continuous flow system with waste material in constant motion between containers such as reservoir 16, heater 12, bioreactor 10, equalization tank 14 if applicable, and so forth. Flow rates in the system can vary depending on retention time desired in any particular container. For example, in preferred embodiments, retention time in bioreactor 10 is between about 6 and 12 hours. To meet this retention time, the flow rate of passage 18 and effluent passage 38 are adjustable as known in the art so that waste material, on average, stays in bioreactor 10 for this period of time. In preferred embodiments, pump 26 also enables conveyance from heater 12 to bioreactor 10 through passage 18. In alternate embodiments, an additional pumps can be specifically operably related to passage 18.

The waste material is conveyed through passage 18 having a first and second end, wherein passage 18 provides entry access to the bioreactor at a first end of passage 18 and providing removal access to the heater at a second end of passage 18. Any type of passage known in the art can be used, such as a pipe or flexible tube. The transport passage may abut or extend within the bioreactor and/or the heater. Passage 18 can generally provide access into bioreactor 10 at any location along the bioreactor. However, in preferred embodiments, passage 18 provides access at an upper portion of bioreactor 10.

Bioreactor 10 provides an anaerobic environment conducive for nonparaffinophilic microorganisms to metabolize waste material, wherein the nonparaffinophilic microorganisms grow and form biofilm on coated substrates. While the bioreactor is beneficial to the growth of nonparaffinophilic microorganisms and the corresponding metabolism of waste material by the nonparaffinophilic microorganisms, it is preferably restrictive to the proliferation of methanogens, wherein methanogens are microorganisms that metabolize carbon dioxide and hydrogen to produce methane and water.

Bioreactor 10 can be any receptacle known in the art for carrying an organic feed material. Bioreactor 10 is anaerobic and therefore substantially airtight. Bioreactor 10 itself may contain several openings. However, these openings are covered with substantially airtight coverings or connections, such as passage 18, thereby keeping the environment in bioreactor 10 substantially anaerobic. Generally, the receptacle will be a limiting factor in the amount of material that can be processed. Therefore, the size and shape of the bioreactor can vary widely within the spirit of the invention depending on output desired and location limitations.

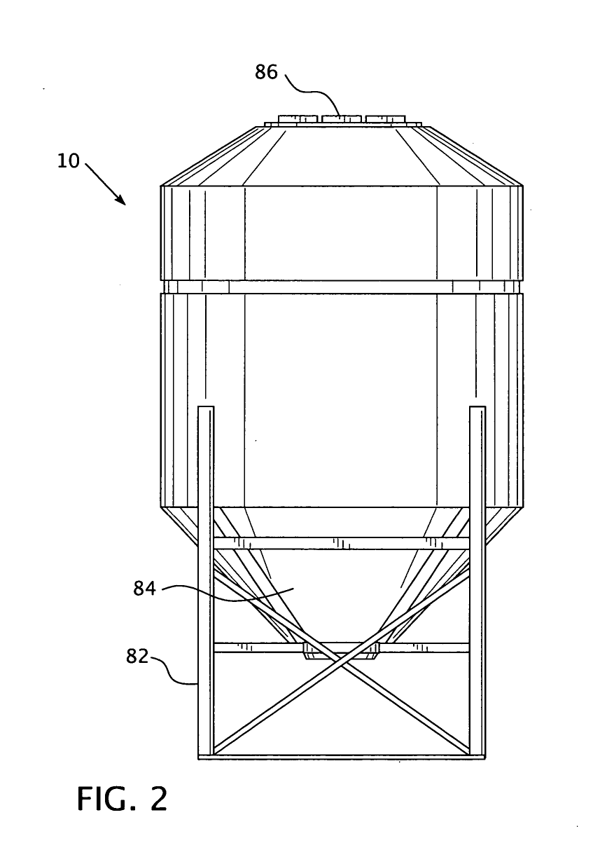

A preferred embodiment of a bioreactor is shown in FIG. 2. Bioreactor 10 can be formed of any material suitable for holding waste material that can further create an airtight, anaerobic environment. In the present invention, bioreactor 10 is constructed of high density polyethylene materials. Other materials, including but not limited to metals or other plastics, can similarly be used. Generally silo-shaped bioreactor 10 has about a 300 gallon capacity with a generally conical bottom 84. Stand 82 is adapted to hold cone bottom 84 and thereby hold bioreactor 10 in all upright position. The bioreactor 10 preferably includes one or a multiplicity of openings that provide a passage for supplying or removing contents from within the bioreactor. The openings may further contain coverings known in the art that cover and uncover the openings as desired. For example, bioreactor 10 preferably includes a central opening covered by lid 86. In alternate embodiments of the invention, the capacity of bioreactor 10 can be readily scaled upward or downward depending on needs or space limitations.

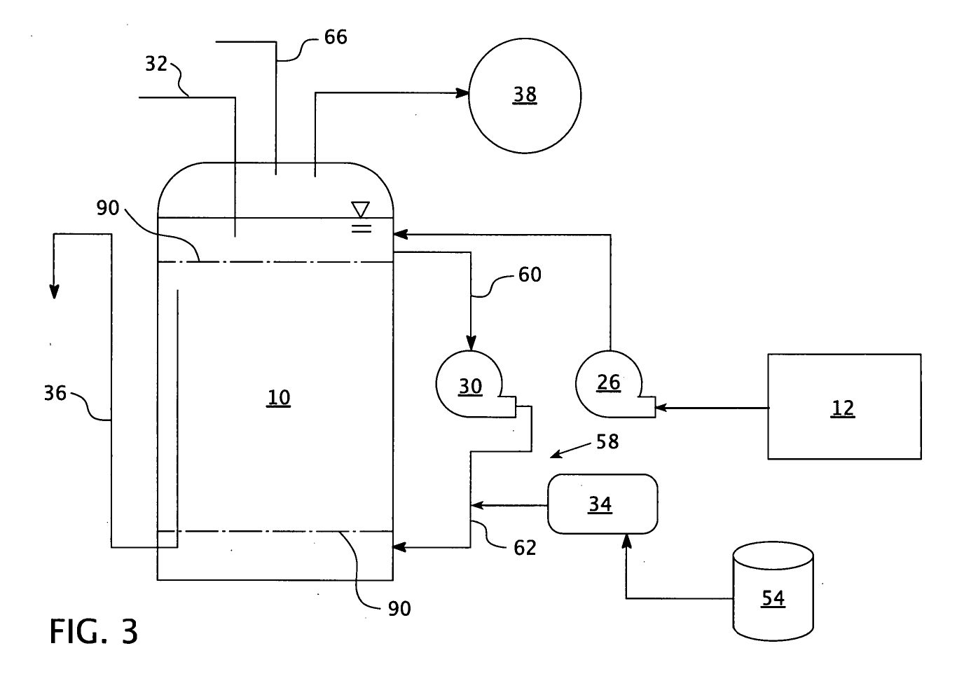

Bioreactor 10 preferably provides a system to remove excess waste material, as shown in FIGS. 1 and 3. In the present embodiment, bioreactor 10 includes effluent passage 36 having an open first and second end that provides a passage from inside bioreactor 10 to outside the bioreactor. The first end of effluent passage 36 may abut bioreactor 10 or extend into the interior of bioreactor 10. If effluent passage 36 extends into the interior of passage 10, the effluent tube preferably extends upwards to generally upper portion of bioreactor 10. When bioreactor 10 is filled with waste material, the open first end of the effluent passage allows an excess waste material to be received by effluent passage 36. Effluent passage 36 preferably extends from bioreactor 10 into a suitable location for effluent, such as a sewer or effluent container, wherein the excess waste material will be deposited through the open second end.

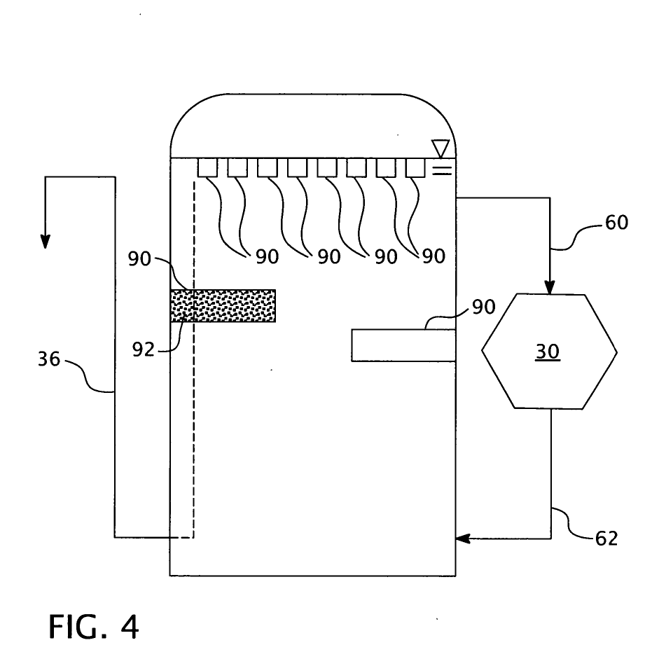

Bioreactor 10 preferably contains one or a multiplicity of substrates 90, as shown in FIG. 4, for providing surface area for attachment and growth of bacterial biofilm. Sizes and shapes of the one or a multiplicity of substrates 90 can vary widely, including but not limited to flat surfaces, pipes, rods, beads, slats, tubes, slides, screens, honeycombs, spheres, object with latticework, or other objects with holes bored through the surface. Numerous substrates can be used, for example, hundreds, as needed.

Substrates 90 preferably are substantially free of interior spaces that potentially fill with gas. In one preferred embodiment, the bioreactor comprises about numerous pieces of floatable 1″ plastic media to provide surface area for attachment of the bacterial biofilm, for example, Flexiring™ Random Packing (Koch-Glitsch.) Some substrates 90 may further be retained below the liquid surface by a perforated acrylic plate.

A carbon-based baiting material 92 is provided within bioreactor 10 as shown FIG. 4. The carbon based material is preferably coated on the one or a multiplicity of substrates 90 within bioreactor 10. The coating baits nonparaffinophilic microorganisms contained in the waste material, which then grow thereon, forming biofilm.

Carbon based baiting material 92 is preferably a gelatinous matrix having at least one carbon compound. In one embodiment, the gelatinous matrix is agar based. In this embodiment, the gelatinous matrix is prepared by placing agar and a carbon compound into distilled water, wherein the agar is a gelatinous mix, and wherein any other gelatinous mix known in the art can be used in place of or in addition to agar within the spirit of the invention.

The carbon compound used with the gelatinous mix to form the gelatinous matrix can vary widely within the spirit of the invention. The carbon source is preferably selected from the group consisting of: glucose, fructose, glycerol, mannitol, asparagines, casein, adonitol. l-arabinose, cellobiose, dextrose, dulcitol, d-galactose, inositol, lactose, levulose, maltose, d-mannose, melibiose, raffinose, rhamnose, sucrose, salicin, d-sorbitol, d-xylose or any combination thereof. Other carbon compounds known in the art, however, can be used within the spirit of the invention.

Generally, the matrix is formed by adding a ratio of three grams of carbon compound and two grams of agar per 100 mL of distilled water. This ratio can be used to form any amount of a mixture up to or down to any scale desired. Once the correct ratio of carbon compound, agar and water are mixed, the mixture is boiled and steam sterilized to form a molten gelatinous matrix.

Substrates 90 can be coated by coating material 92 by hand, by machine or by any means known in the art. In one embodiment, the carbon based coating material 92 may be coated directly onto the substrate. In alternative embodiments, however, an adhesive layer may be located between the carbon based coating material 92 and the substrate, the adhesive being any adhesive known in the art for holding carbon based compounds. In a preferred embodiment, the adhesive includes a plurality of gel beads, wherein carbon based coating material 92 is affixed to the gel beads ironically or by affinity.

For example, substrate 90 with the gelatinous matrix containing a carbon source can be prepared by the following method. A receptacle, such as a laboratory beaker, is first filled with 100 ml of distilled water. Placed into the beaker are two (2) grams of agar (the gelatinous matrix) per three (3) grams of a carbon source (such as glucose). This mixture is then boiled and steam sterilized and the molten gelatinous matrix with a carbon source is poured into a receptacle sitting on a hot plate. In this way the gelatinous matrix/carbon source remains molten. After this, a substrate 90 is dropped into the molten gelatinous matrix/carbon source and becomes coated therewith. The now coated slide is removed from the petri dish and allowed to stand for a minute or two in order to solidify the coating thereon. The slide with the coating of a gelatinous matrix containing a carbon source is then ready to be placed in bioreactor 10.

An alternative method of preparing the coated substrate involves first coating the substrate with an adhesive, such as collodion and then applying a plurality of gel beads (commercially available from Pharmacia of Parsippany, N.J.) to the adhesive. The gel beads are approximately one micron in diameter. The coating containing the coating of gel beads is now immersed in a buffering agent containing the carbon source (such as glucose) to attach the carbon source to the gel beads either ionically or affinity-wise.

In alternate embodiments, the one or a multiplicity of substrates 90 are generally inserted into the bioreactor through corresponding slots, such that the substrates can be added or removed from the bioreactor without otherwise opening the bioreactor. In further alternate embodiments, the substrates are affixed to an interior surface of the bioreactor.

In an additional embodiment, coating material 92 is conveyed from the container holding carbon based coating material 92 into a hollow or partially hollow interior channel of the substrate. The gelatinous matrix is conveyed into the channel with a conveying device, preferably a pump. The conveying device can be any pumping means known in the art, including hand or machine. The carbon based coating material 92 permeates from the channel of the substrate to the exterior through the holes, coating the substrate surface. The carbon based coating material 92 on the substrate can be continually replenished at any time by conveying more gelatinous matrix into the interior of the substrate. The flow of carbon based coating material 92 can be regulated by the conveying device such that the substrate is coated and/or replenished at any speed or rate desired. Further, the entire substrate need not be covered by the carbon based coating material 92, although preferably the majority of the substrate is covered at any moment in time.

In this embodiment, the invention may further provide a method for producing hydrogen and isolating microorganisms having anaerobic bioreactor for holding waste material, one or a multiplicity of substrates contained within the bioreactor, the one or a multiplicity of substrates having a coating disposed thereon for hosting the growth of biofilm, wherein the coating is a replenishable coating from a coating source outside the bioreactor. The coating is contained in a coating container or other container proximate the bioreactor. The system further contains a passage connecting the coating container and the interior channel of one or a multiplicity of substrates. Coating is pumped from the coating container through the passage and into the channel, where the coating permeates from the channel through a permeable or semi-permeable surface of the substrates. As the coating permeates to the surface, it replenishes, i.e., supplements or replaces, coatings already present on the substrates. Alternatively, if no coating is present, the coating permeates to provide an initial coating on the substrates. By replenishing coating, the system has a continuous supply of bait and feeding material for nonparaffinophilic microorganisms. The nonparaffinophilic microorganisms for biofilm on the coated substrates and are thereby isolated on the substrates.

The substrate provides an environment for the growth of nonparaffinophilic microorganisms in the bioreactor. This is advantageous as substrates enable microorganisms to obtain more nutrients and expend less energy than a similar microorganism floating loosely in waste material.

Further, nonparaffinophilic microorganisms that can metabolize the waste material grow quickest. The microorganisms, baited by the carbon based coating material, attach themselves to the substrate, thereby forming a slime layer on the substrate generally referred to as a biofilm. The combination of carbon based coating material 92 on the substrate, waste material and the environmental conditions not favorable to methanogens allows the nonparaffinophilic microorganisms to grow, multiply and form biofilm on the substrate. If the nonparaffinophilic microorganisms metabolize the waste, the biofilm, supported by the coated substrate, can thrive.

In order to increase growth and concentration on the substrate coated with a carbon based baiting means for nonparaffinophilic microorganisms, the surface area of the substrate can be increased. Increasing the surface area can be achieved by optimizing the surface area of a single substrate within the bioreactor, adding a multiplicity of substrates within the bioreactor, or a combination of both.

Colonies of nonparaffinophilic microorganisms growing on substrates 90, or elsewhere in the bioreactor, can be selected from the biofilm wherein selecting means removing one or a multiplicity of colonies from the biofilm from the bioreactor 10 by a method known in the art. The selected colonies, by successfully growing in and metabolizing the waste material, are good candidates for use in a bioremediation or biodegradation process of the waste material. The selected colonies can then be further isolated and/or identified by means methods known in the art. Such as conventional or molecular based systems.

Once identified, the isolated nonparaffinophilic microorganisms can be used as microorganisms for the biodegradation or bioremediation of waste products. The nonparaffinophilic microorganism can be scaled up and added to additional waste material in an anaerobic environment outside of system 100 to break down the waste material in and produce gases that are substantially non-methane. The isolated nonparaffinophilic microorganism can be scaled up by methods known in the art. For example, the isolated nonparaffinophilic microorganism can be grown in a suitable broth and then centrifuged to remove a potion of the broth, thereby resulting in concentrated amount of the isolated nonparaffinophilic microorganism. The concentrated amount of nonparaffinophilic microorganism can then be added to the additional waste material. Ideally, the additional waste material would be maintained at a pH level between about 3.5 and 6.0 pH.

Bioreactor 10 may further include a coating of alginate within the interior of the bioreactor. The thickness and type of alginate coating can vary within the bioreactor. Thus, the bioreactor may have levels of alginate. i.e., areas of different formulations and amounts of alginate in different locations within the bioreactor.

In further embodiments, a directional flow is achieved in bioreactor 10. Circulation system 58 is provided in operable relation to bioreactor 10. Circulation system 58 enables circulation of waste material contained within bioreactor 10 by removing waste material at one location in bioreactor 10 and reintroduces the removed waste material at a separate location in bioreactor 10, thereby creating a directional flow in the bioreactor. The directional flow aids the microorganisms within the waste material in finding waste materials and substrates on which to grown biofilm. As could be readily understood, removing waste material from a lower region of bioreactor 10 and reintroducing it at an upper region of bioreactor 10 would create a downward flow in bioreactor 10. Removing waste material from an upper region of bioreactor 10 and reintroducing it at a lower region would create an up-flow in bioreactor 10.

In preferred embodiments, as shown in FIG. 1, circulation system 58 is arranged to produce an up-flow of any waste material contained in bioreactor 10. Passage 60 provides removal access at a higher point than entry access provided is provided by passage 62. Pump 30 facilitates movement from bioreactor 10 into passage 60, from passage 60 into passage 62, and from passage 62 back into bioreactor 10, creating up-flow movement in bioreactor 10. Pump 30 can be any pump known in the art for pumping organic feed material. In preferred embodiments, pump 30 is an air driven centrifugal pump. Other arrangements can be used, however, while maintaining the spirit of the invention. For example, a pump could be operably related to a single passage that extends from one located of the bioreactor to another.

One or a multiplicity of additional treatment steps can be performed on the waste material, either in bioreactor 10 or elsewhere in the system, for the purpose of making the waste material more conducive to proliferation of nonparaffinophilic microorganisms. The one or a multiplicity of treatment steps include, but are not limited to, aerating the waste material, diluting the waste material with water or other dilutant, controlling the pH of the waste material, adjusting electrolyte contents (Na, K, Cl, Mg, Ca, etc.) and adding additional chemical compounds to the waste material. Additional chemical compounds added by treatment apparatuses include anti-fungal agents, phosphorous supplements, yeast extract or nonparaffinophilic microorganisms inoculation. The apparatus performing these treatment steps can be any apparatuses known in the art for incorporating these treatments. For example, in one embodiment, a dilution apparatus is a tank having a passage providing controllable entry access of a dilutant, such as water, into bioreactor 10. In some preferred embodiments, the treatment steps are performed in circulation system 58. In other embodiments, treatment steps of the same type may be located at various points in the bioreactor system to provide treatments at desired locations.

Keeping waste material contained within bioreactor 10 within a favorable pH range is conducive to lack of methane production. In preferred embodiments, pH controller 34 monitors the pH level of contents contained within bioreactor 10. In preferred embodiments, the pH of the waste material in bioreactor 10 is maintained between about 3.5 to 6.0 pH, most preferably between about 4.5 to 5.5 pH, as shown in Table 2. In further preferred embodiments, pH controller 34 controllably monitors the pH level of the waste material and adjustably controls the pH of the organic feed material if the waste material falls out of or is in danger of falling out of the desired range. As shown in FIG. 1, pH controller 34 monitors the pH level of contents contained in passage 62, such as waste material, with a pH sensor (represented as the wavy line connecting pH controller 34 and passage 62.) As could readily be understood, pH controller 34 can be operably related to any additional or alternative location that potentially holds waste material, for example, passage 60, 62 or bioreactor 10 as shown in FIG. 3. Controlling pH in the bioreactor may be performed alternatively or additionally to heating waste material prior to introduction into the bioreactor.

If the pH of the waste material falls out of a desired range, the pH is preferably adjusted back into the desired range. Control of a pH level provides an environment that enables at least some nonparaffinophilic microorganisms to function while similarly providing an environment unfavorable to methanogens. Control of pH of the waste material in the bioreactor can be achieved by any means known in the art. In one embodiment, a pH controller 34 monitors the pH and can add a pH control solution from container 54 in an automated manner if the pH of the organic feed material moves out of a desired range. In a preferred embodiment, the pH monitor controls the organic feed material's pH through automated addition of a sodium or potassium hydroxide solution. One such apparatus for achieving this is an Etatron DLX pH monitoring device. Preferred ranges of pH for the organic feed material is between about 3.5 and 6.0, with a more preferred range between about 4.0 and 5.5 pH.

In one embodiment, the wastewater is a grape juice solution prepared using Welch's Concord Grape Juice™ diluted in chlorine-free tap water at approximately 32 mL of juice per Liter. Alternatively, the solution is aerated previously for 24 hours to substantially remove chlorine. Due to the acidity of the juice, the pH of the organic feed material is typically around 4.0. The constitutional make-up of the grape juice solution is shown in Table 1.

TABLE 1
Composition of concord grape juice. Source:
Welch's Company, personal comm., 2005.

Concentration (unit indicated)

| Constituent | Mean | Range |
|---|---|---|
| Carbohydrates¹ | | 15-18% |
| glucose | 6.2% | 5-8% |
| fructose | 5.5% | 5-8% |
| sucrose | 1.8% | 0.2-2.3% |
| maltose | 1.9% | 0-2.2% |
| sorbitol | 0.1% | 0-0.2% |
| Organic Acids¹ | | 0.5-1.7% |
| Tartaric acid | 0.84% | 0.4-1.35% |
| Malic acid | 0.86% | 0.17-1.54% |
| Citric acid | 0.044% | 0.03-0.12% |
| Minerals¹ | | |
| Calcium | | 17-34 mg/L |
| Iron | | 0.4-0.8 mg/L |
| Magnesium | | 6.3-11.2 mg/L |
| Phosphorous | | 21-28 mg/L |
| Potassium | | 175-260 mg/L |
| Sodium | | 1-5 mg/L |
| Copper | | 0.10-0.15 mg/L |
| Manganese | | 0.04-0.12 mg/L |
| Vitamins¹ | | |
| Vitamin C | | 4 mg/L |
| Thiamine | | 0.06 mg/L |
| Riboflavin | | 0.04 mg/L |
| Niacin | | 0.2 mg/L |
| Vitamin A | | 80 I.U. |
| pH | | 3.0-3.5 |
| Total solids | | 18.5% |

¹additional trace constituents in these categories may be present.

Bioreactor 10 further preferably includes an overflow cut-off switch 66, as shown in FIG. 3, to turn off feed pump 26 if the organic feed material exceeds or falls below a certain level in the bioreactor.

Exhaust system 70 exhausts gas produced by the nonparaffinophilic microorganisms. Any exhaust system known in the art can be used. In a preferred embodiment, as shown in FIG. 1, exhaust system includes exhaust passage 72, backflow preventing device 74, gas flow measurement and totalizer 76, air blower 46 and exhaust pipe 78.

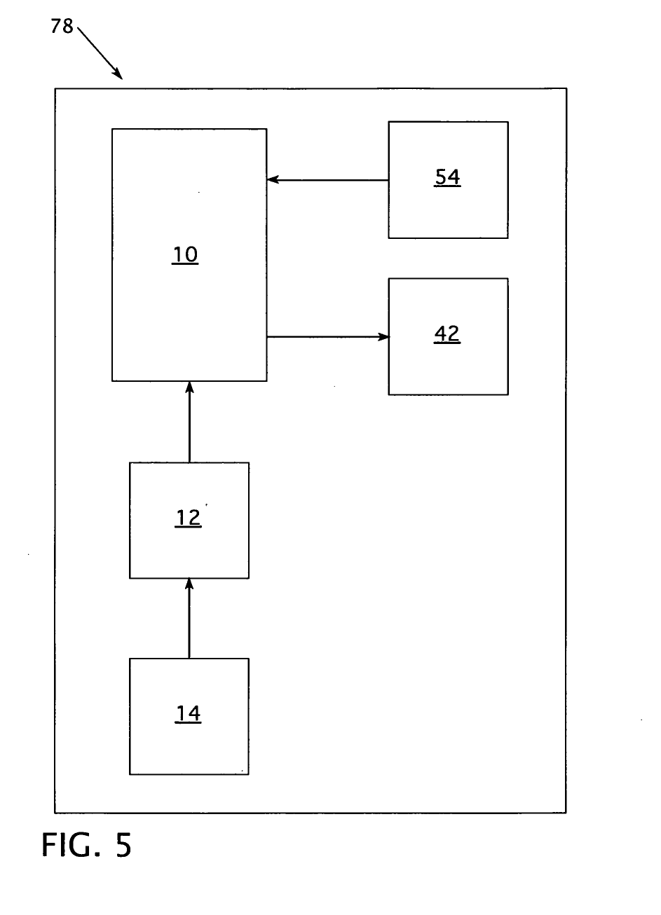

The entire method may be housed in a single housing unit 78 is shown in FIG. 5. The containers and bioreactors will be filled with liquid and thus will be heavy. For example, if a 300 gallon cone-bottom bioreactor is used, the bioreactor can weigh about 3,000 lbs. The stand preferably has four legs, with a 2″ steel plate tying the legs together. If it is assumed that each leg rests on a 2×2 square, then the loading to the floor at those spots would be 190 lbs/sq inch. The inside vertical clearance is preferably at least 84 inches. For safety reasons, the main light switch for the building will be mounted on the outside next to the entry door and the electrical panel will be mounted on the exterior of the building so that all power to the building could be cut without entering. In this further preferred embodiment, the system is preferably proximate to industrial facility.

All plumbing connections for the system are water tight, and the gas-side connections are pressure checked. Once the produced gas has been scrubbed of CO2, it will pass through a flow sensor and then be exhausted to the atmosphere through a stand pipe. A blower (as used in boats where gas fumes might be present) will add air to the stand pipe at a rate of more than 500 to 1, thus reducing possible hydrogen concentration well below the LEL. As soon as this mixture reaches the top of the pipe, it will be dissipated by the atmosphere.

In case of a hydrogen leak inside the building, the housing unit preferably includes a hydrogen sensor connected to a relay which will activate an alarm and a ventilation system. The ventilation system is preferably mounted on the outside of the building and will force air through the building and out the roof vents. The hydrogen sensor is preferably set to activate if the hydrogen concentration reaches even 25% of the LEL. The only electrical devices will be a personal computer, low-voltage sensors, electrical outlets and connections, all of which will be mounted on the walls lower than normal. The hydrogen sources will preferably be located high in the room and since hydrogen does not settle.

### EXAMPLE 1

A multiplicity of bioreactors house waste materials were initially operated at pH 4.0 and a flow rate of 2.5 mL min⁻¹, resulting in a hydraulic retention time (HRT) of about 13 h (0.55 d). This is equivalent to a dilution rate of 1.8 d⁻¹. After one week all six bioreactors were at pH 4.0, the ORP ranged from −300 to −450 mV, total gas produced by biodegradation averaged 1.6 L d⁻¹ including hydrogen production averaged 0.8 L d⁻¹. The mean COD of the waste material during this period was 4,000 mg L⁻¹ and the mean effluent COD was 2.800 mg L⁻¹, for a reduction of 30%. After one week, the pHs of certain bioreactors were increased by one half unit per day until the six bioreactors were established at different pH levels ranging from 4.0 to 6.5. Over the next three weeks at the nests pH settings, samples were collected and analyzed each weekday. It was found that the optimum for gas production through biodegradation in this embodiment was pH 5.0 at 1.48 L hydrogen d⁻¹ (Table 2).

TABLE 2
Production of hydrogen in 2-L anaerobic
bioreactors as a function of pH.

| | Total gas | H2 | H2 | H2 per Sugar |
|---|---|---|---|---|
| pH | L/day | L/day | L/g COD | moles/mole |
| 4.0^{a} | 1.61 | 0.82 | 0.23 | 1.81 |
| 4.5^{b} | 2.58 | 1.34 | 0.23 | 1.81 |
| 5.0^{c} | 2.74 | 1.48 | 0.26 | 2.05 |
| 5.5^{d} | 1.66 | 0.92 | 0.24 | 1.89 |
| 6.0^{d} | 2.23 | 1.43 | 0.19 | 1.50 |
| 6.5^{e} | 0.52 | 0.31 | 0.04 | 0.32 |

^{a}mean of 20 data points
^{b}mean of 14 data points
^{c}mean of 11 data points
^{d}mean of 7 data points
^{e}mean of 6 data points

The complete data set is provided in Tables 3a and 3b.

Samples of biogas were analyzed several times per week from the beginning of the study, initially using a Perkin Elmer Autosystem GC with TCD, and then later with a Perkin Elmer Clarus 500 GC with TCD in series with an FID. Methane was not detected with the TCD, but trace amounts were detected with the FID (as much as about 0.05%).

Over a ten-day period, the waste material was mixed with sludge obtained from a methane-producing anaerobic digester at a nearby wastewater treatment plant at a rate of 30 mL of sludge per 20 L of diluted grape juice. There was no observed increase in the concentration of methane during this period. Therefore, it was concluded that the preheating of the feed to about 65° C. as described previously was effective in deactivating the microorganisms contained in the sludge. Hydrogen gas production rate was not affected (data not shown).

Using this example, biodegradation of waste material is generated using a nonparaffinophilic microorganisms. Under these conditions, using plastic packing material to retain microbial biomass, a hydraulic residence time of about 0.5 days resulted in the generation of about 0.75 volumetric units of hydrogen gas per unit volume of bioreactor per day.

Colonies of nonparaffinophilic microorganisms were selected from the biofilms in the bioreactor and identified using standard microbiologic methodology for bacterial identification. The identified nonparaffinophilic microorganisms can then be used to biodegradation or bioremediation process for new, additional waste materials.

Whereas particular embodiments of this invention have been described above for purposes of illustration, it will be evident to those skilled in the art that numerous variations of the details of the present invention may be made without departing from the invention as defined in the appended claims.

TABLE 3a
Bioreactor Operating Data

| | | | | | | | | | | COD | | | | | | | |
|---|---|---|---|---|---|---|---|---|---|---|---|---|---|---|---|---|---|
| | | | GAS | | Liquid Readings | | | | | | Ef- | Re- | | | Performance | | |
| | | col- | Tot | after | Ef- | | | | | | flu- | mov- | | | Total | | |
| | | lec- | vol- | scrub- | flu- | | Net | | | Feed | ent | al | Load- | Con- | gas | H2 | H2 |
| | Reac- | tion | ume | bing | ent | NaOH | Feed | | | (mg/ | (mg/ | (mg/ | ing | sumed | L/ | L/ | L/g |
| Date | tor | hours | (mL) | (mL) | (mL) | (mL) | (mL) | ORP | pH | L) | L) | L) | (g) | (g) | day | day | COD |
| 17-Nov | C | 5.5 | 360 | 200 | 840 | 120 | 720 | −344 | 4.9 | 4,907 | 2,880 | 2,027 | 3.533 | 1.459 | 1.57 | 0.87 | 0.14 |
| 18-Nov | C | 5 | 370 | 200 | 1120 | 70 | 1050 | −328 | 4.9 | 3,680 | 2,480 | 1,200 | 3.864 | 1.260 | 1.78 | 0.96 | 0.16 |
| 29-Nov | C | 4.25 | 415 | 200 | 920 | 50 | 870 | −403 | 4.9 | 5,013 | 3,093 | 1,920 | 4.362 | 1.670 | 2.34 | 1.13 | 0.12 |
| 17-Nov | E | 5.5 | 490 | 270 | 1210 | 115 | 1095 | −352 | 5.0 | 4,907 | 4,747 | 160 | 5.373 | 0.175 | 2.14 | 1.18 | 1.54 |
| 1-Dec | D | 3.5 | 540 | 250 | 710 | 85 | 625 | −395 | 5.0 | 5,173 | 3,573 | 1,600 | 3.233 | 1.000 | 3.70 | 1.71 | 0.25 |
| 17-Nov | F | 5.5 | 475 | 225 | 1120 | 130 | 990 | −367 | 5.0 | 4,907 | 3,760 | 1,147 | 4.858 | 1.135 | 2.07 | 0.98 | 0.20 |
| 5-Dec | D | 4.5 | 580 | 310 | 710 | 77 | 633 | −423 | 5.0 | 4,267 | 3,573 | 694 | 2.701 | 0.439 | 3.09 | 1.65 | 0.71 |
| 6-Dec | D | 3 | 450 | 240 | 490 | 43 | 447 | −420 | 5.0 | 4,853 | 3,253 | 1,600 | 2.169 | 0.715 | 3.60 | 1.92 | 0.34 |
| 17-Nov | D | 3.5 | 680 | 415 | 580 | 83 | 497 | −326 | 5.0 | 4,907 | 4,213 | 694 | 2.439 | 0.345 | 4.66 | 2.85 | 1.20 |
| 2-Dec | D | 3.75 | 640 | 340 | 830 | 66 | 764 | −412 | 5.0 | 4,587 | 3,787 | 800 | 3.504 | 0.611 | 4.10 | 2.18 | 0.56 |
| 22-Nov | C | 3.75 | 460 | 295 | 800 | 50 | 750 | −349 | 5.0 | 4,107 | 1,280 | 2,827 | 3.080 | 2.120 | 2.94 | 1.89 | 0.14 |
| averages | | 4.34 | 496 | 268 | 848 | 81 | 767 | −374.5 | 5.0 | 4,664 | 3,331 | 1,333 | 3.579 | 1.023 | 2.74 | 1.48 | 0.26 |
| 5-Dec | C | 4.5 | 470 | 250 | 900 | 103 | 797 | −429 | 5.4 | 4,267 | 3,413 | 854 | 3.401 | 0.680 | 2.51 | 1.33 | 0.37 |
| 18-Nov | F | 5 | 90 | 45 | 600 | 55 | 545 | −451 | 5.5 | 3,680 | 3,440 | 240 | 2.006 | 0.131 | 0.43 | 0.22 | 0.34 |
| 21-Nov | D | 4 | 130 | 70 | 830 | 80 | 750 | −454 | 5.5 | 3,493 | 3,360 | 133 | 2.620 | 0.100 | 0.78 | 0.42 | 0.70 |
| 22-Nov | D | 3.75 | 360 | 250 | 766 | 69 | 696 | −461 | 5.5 | 4,107 | 2,880 | 1,227 | 2.858 | 0.854 | 2.30 | 1.60 | 0.29 |
| 29-Nov | D | 4.25 | 100 | 50 | 940 | 100 | 840 | −456 | 5.5 | 5,013 | 3,307 | 1,707 | 4.211 | 1.434 | 0.56 | 0.28 | 0.03 |
| 2-Dec | C | 3.75 | 560 | 290 | 810 | 93 | 717 | −430 | 5.5 | 4,587 | 3,573 | 1,014 | 3.289 | 0.727 | 3.52 | 1.86 | 0.40 |
| 6-Dec | C | 3 | 250 | 130 | 570 | 45 | 525 | −428 | 5.5 | 4,853 | 3,627 | 1,226 | 2.548 | 0.644 | 2.00 | 1.04 | 0.20 |
| averages | | 4.04 | 279 | 155 | 774 | 78 | 696 | −444.1 | 5.5 | 4,286 | 3,371 | 914 | 2.982 | 0.636 | 1.66 | 0.92 | 0.24 |
| 21-Nov | E | 4 | 360 | 250 | 930 | 130 | 800 | −400 | 6.0 | 3,493 | 2,987 | 506 | 2.794 | 0.405 | 2.10 | 1.50 | 0.62 |
| 22-Nov | E | 3.75 | 380 | 280 | 820 | 127 | 693 | −411 | 6.0 | 4,107 | 2,453 | 1,653 | 2.846 | 1.146 | 2.43 | 1.79 | 0.24 |
| 29-Nov | E | 4.25 | 360 | 230 | 870 | 71 | 799 | −467 | 6.0 | 5,013 | 1,973 | 3,040 | 4.006 | 2.429 | 2.03 | 1.30 | 0.09 |
| 1-Dec | E | 3.5 | 420 | 250 | 770 | 127 | 643 | −471 | 6.0 | 5,173 | 2,933 | 2,240 | 3.326 | 1.440 | 2.88 | 1.71 | 0.17 |
| 2-Dec | E | 3.75 | 280 | 170 | 540 | 85 | 455 | −443 | 6.0 | 4,587 | 3,360 | 1,227 | 2.087 | 0.558 | 1.79 | 1.09 | 0.30 |
| 5-Dec | E | 4.5 | 410 | 240 | 930 | 156 | 774 | −487 | 6.0 | 4,267 | 3,253 | 1,014 | 3.303 | 0.785 | 2.19 | 1.28 | 0.31 |
| 6-Dec | E | 3 | 380 | 170 | 660 | 105 | 555 | −490 | 6.0 | 4,853 | 2,293 | 2,560 | 2.693 | 1.421 | 2.24 | 1.36 | 0.12 |
| averages | | 3.82 | 354 | 227 | 789 | 114 | 674 | −453 | 6.0 | 4,499 | 2,750 | 1,749 | 3.033 | 1.179 | 2.23 | 1.43 | 0.19 |
| 29-Nov | F | 4.25 | 90 | 45 | 870 | 150 | 720 | −501 | 6.5 | 5,013 | 1,707 | 3,307 | 3.610 | 2.381 | 0.51 | 0.25 | 0.02 |
| 2-Dec | F | 3.75 | 20 | 0 | 810 | 136 | 674 | −497 | 6.5 | 4,587 | 3,573 | 1,014 | 3.092 | 0.683 | 0.13 | 0.00 | 0.00 |
| 22-Nov | F | 3.75 | 120 | 105 | 790 | 128 | 662 | −477 | 6.5 | 4,107 | 2,240 | 1,867 | 2.719 | 1.236 | 0.77 | 0.67 | 0.08 |
| 5-Dec | F | 4.5 | 10 | 0 | 670 | 121 | 549 | −532 | 6.5 | 4,267 | 2,827 | 1,440 | 2.343 | 0.791 | 0.05 | 0.00 | 0.00 |
| 6-Dec | F | 3 | 60 | 50 | 480 | 90 | 390 | −515 | 6.5 | 4,853 | 2.240 | 2,613 | 1.893 | 1.019 | 0.48 | 0.40 | 0.05 |
| 21-Nov | F | 4 | 200 | 100 | 910 | 150 | 760 | −472 | 6.5 | 3,493 | 2,613 | 880 | 2.655 | 0.669 | 1.20 | 0.60 | 0.15 |
| averages | | 3.88 | 83 | 50 | 755 | 129 | 626 | −499 | 6.5 | 4,387 | 2,533 | 1,853 | 2.745 | 1.160 | 0.52 | 0.31 | 0.04 |

TABLE 3b
Bioreactor Operating Data Continued.

| | | | | | | | | | | COD | | | | | | | |
|---|---|---|---|---|---|---|---|---|---|---|---|---|---|---|---|---|---|
| | | | GAS | | Liquid Readings | | | | | | Ef- | Re- | | | Performance | | |
| | | col- | Total | after | Ef- | | | | | | flu- | mov- | | | Total | | |
| | | lec- | vol- | scrub- | flu- | | Net | | | Feed | ent | al | Load- | Con- | gas | H2 | H2 |
| | Reac- | tion | ume | bing | ent | NaOH | Feed | | | (mg/ | (mg/ | (mg/ | ing | sumed | L/ | L/ | L/g |
| Date | tor | hours | (mL) | (mL) | (mL) | (mL) | (mL) | ORP | pH | L) | L) | L) | (g) | (g) | day | day | COD |
| 14-Nov | A | 5 | 540 | 220 | 780 | 0 | 780 | −408 | 4.0 | 4,480 | 2,293 | 2,187 | 3.494 | 1.706 | 2.59 | 1.06 | 0.13 |
| 14-Nov | B | 5 | 380 | 220 | 840 | 0 | 840 | −413 | 4.1 | 4,480 | 2,453 | 2,027 | 3.763 | 1.702 | 1.82 | 1.06 | 0.13 |
| 14-Nov | C | 5 | 350 | 170 | 870 | 0 | 870 | −318 | 4.1 | 4,480 | 2,293 | 2,187 | 3.898 | 1.902 | 1.68 | 0.82 | 0.09 |
| 14-Nov | D | 5 | 320 | 130 | 920 | 0 | 920 | −372 | 4.1 | 4,480 | 1,920 | 2,560 | 4.122 | 2.355 | 1.54 | 0.62 | 0.06 |
| 14-Nov | E | 5 | 240 | 100 | 920 | 0 | 920 | −324 | 4.3 | 4,480 | 2,773 | 1,707 | 4.122 | 1.570 | 1.15 | 0.48 | 0.06 |
| 14-Nov | F | 5 | 50 | 25 | 810 | 0 | 810 | −329 | 4.0 | 3,307 | 2,080 | 1,227 | 2.679 | 0.994 | 0.24 | 0.12 | 0.03 |
| 15-Nov | A | 5.5 | 450 | 230 | 1120 | 25 | 1095 | −400 | 4.0 | 3,307 | 3,787 | (480) | 3.621 | −0.525 | 1.96 | 1.00 | −0.44 |
| 15-Nov | B | 5.5 | 450 | 235 | 1180 | 35 | 1145 | −384 | 4.0 | 3,307 | 3,253 | 54 | 3.787 | 0.061 | 1.96 | 1.03 | 3.82 |
| 15-Nov | C | 5.5 | 250 | 130 | 640 | 0 | 640 | −278 | 4.0 | 3,307 | 3,520 | (213) | 2.116 | −0.136 | 1.09 | 0.57 | −0.95 |
| 15-Nov | E | 5.5 | 455 | 225 | 1160 | 0 | 1160 | −435 | 4.0 | 3,307 | 3,467 | (160) | 3.836 | −0.185 | 1.99 | 0.98 | −1.21 |
| 15-Nov | F | 5.5 | 430 | 235 | 1160 | 0 | 1160 | −312 | 4.0 | 3,307 | 3,413 | (106) | 3.836 | −0.123 | 1.88 | 1.03 | −1.91 |
| 16-Nov | A | 5 | 380 | 190 | 1020 | 27 | 993 | −414 | 4.0 | 4,693 | 3,627 | 1,066 | 4.660 | 1.059 | 1.82 | 0.91 | 0.18 |
| 5-Dec | A | 4.5 | 200 | 110 | 500 | 35 | 465 | −439 | 4.0 | 4,267 | 4,160 | 107 | 1.984 | 0.050 | 1.07 | 0.59 | 2.21 |
| 18-Nov | A | 5 | 360 | 190 | 200 | 0 | 200 | −423 | 4.0 | 3,680 | 5,227 | (1,547) | 0.736 | −0.309 | 1.73 | 0.91 | −0.61 |
| 21-Nov | A | 4 | 320 | 170 | 800 | 40 | 760 | −429 | 4.0 | 3,493 | 3,680 | (187) | 2.656 | −0.142 | 1.92 | 1.02 | −1.20 |
| 22-Nov | A | 3.75 | 285 | 190 | 725 | 21 | 704 | −432 | 4.0 | 4,107 | 2,293 | 1,813 | 2.891 | 1.277 | 1.82 | 1.22 | 0.15 |
| 29-Nov | A | 4.25 | 310 | 155 | 750 | 24 | 726 | −439 | 4.0 | 5,013 | 3,520 | 1,493 | 3.640 | 1.084 | 1.75 | 0.88 | 0.14 |
| 2-Dec | A | 3.75 | 250 | 120 | 660 | 26 | 634 | −438 | 4.0 | 4,587 | 3,893 | 694 | 2.908 | 0.440 | 1.60 | 0.77 | 0.27 |
| 6-Dec | A | 3 | 150 | 75 | 540 | 0 | 540 | −441 | 4.0 | 4,853 | 3,093 | 1,760 | 2.621 | 0.950 | 1.20 | 0.60 | 0.08 |
| 17-Nov | A | 5.5 | 300 | 160 | 1010 | 30 | 980 | −414 | 4.0 | 4,907 | 3,520 | 1,387 | 4.809 | 1.359 | 1.31 | 0.70 | 0.12 |
| averages | | 4.81 | 324 | 164 | 830 | 13 | 817 | −392 | 4.0 | 4,092 | 3,213 | 879 | 3.344 | 0.718 | 1.61 | 0.82 | 0.23 |
| 16-Nov | B | 5 | 400 | 200 | 1125 | 45 | 1080 | −397 | 4.5 | 4,693 | 3,520 | 1,173 | 5.068 | 1.267 | 1.92 | 0.96 | 0.16 |
| 16-Nov | D | 5 | 400 | 165 | 960 | 60 | 900 | −360 | 4.5 | 4,693 | 3,573 | 1,120 | 4.224 | 1.008 | 1.92 | 0.79 | 0.16 |
| 16-Nov | E | 5 | 490 | 240 | 1100 | 72 | 1028 | −324 | 4.5 | 4,693 | 3,413 | 1,280 | 4.824 | 1.315 | 2.35 | 1.15 | 0.18 |
| 1-Dec | B | 3.5 | 500 | 260 | 570 | 45 | 525 | −415 | 4.5 | 5,173 | 3,680 | 1,493 | 2.716 | 0.784 | 3.43 | 1.78 | 0.33 |
| 6-Dec | B | 3 | 470 | 240 | 650 | 40 | 610 | −411 | 4.5 | 4,853 | 3,360 | 1,493 | 2.960 | 0.911 | 3.76 | 1.92 | 0.26 |
| 21-Nov | B | 4 | 560 | 300 | 930 | 50 | 880 | −397 | 4.5 | 3,493 | 3,147 | 346 | 3.074 | 0.305 | 3.36 | 1.80 | 0.98 |
| 2-Dec | B | 3.75 | 640 | 320 | 830 | 50 | 780 | −407 | 4.5 | 4,587 | 3,413 | 1,174 | 3.578 | 0.915 | 4.10 | 2.05 | 0.35 |
| 17-Nov | B | 5.5 | 450 | 220 | 1165 | 50 | 1115 | −406 | 4.5 | 4,907 | 2,933 | 1,974 | 5.471 | 2.201 | 1.96 | 0.96 | 0.10 |
| 18-Nov | B | 5 | 390 | 220 | 860 | 42 | 818 | −406 | 4.5 | 3,680 | 2,960 | 720 | 3.010 | 0.589 | 1.87 | 1.06 | 0.37 |
| 22-Nov | B | 3.75 | 585 | 395 | 835 | 50 | 785 | −397 | 4.5 | 4,107 | 2,720 | 1,387 | 3.224 | 1.089 | 3.74 | 2.53 | 0.36 |
| 29-Nov | B | 4.25 | 620 | 320 | 920 | 42 | 878 | −410 | 4.5 | 5,013 | 3,307 | 1,707 | 4.402 | 1.498 | 3.50 | 1.81 | 0.21 |
| 5-Dec | B | 4.5 | 390 | 190 | 750 | 37 | 713 | −417 | 4.5 | 4,267 | 3,840 | 427 | 3.042 | 0.304 | 2.08 | 1.01 | 0.62 |
| 16-Nov | F | 5 | 400 | 200 | 1082 | 93 | 989 | −324 | 4.5 | 4,693 | 3,093 | 1,600 | 4.641 | 1.582 | 1.92 | 0.96 | 0.13 |
| 16-Nov | C | 5 | 400 | 200 | 950 | 74 | 876 | −325 | 4.6 | 4,693 | 2,933 | 1,760 | 4.111 | 1.541 | 1.92 | 0.96 | 0.13 |
| averages | | 4.45 | 478 | 248 | 909 | 54 | 856 | −385 | 4.5 | 4,539 | 3,278 | 1,261 | 3.883 | 1.079 | 2.58 | 1.34 | 0.23 |

### SELECTED CITATIONS AND BIBLIOGRAPHY

Brosseau, J. D. and J. E. Zajic. 1982a. Continuous Microbial Production of Hydrogen Gas. Int. J. Hydrogen Energy 7(8): 623-628.
Brosseau, J. D. and J. E. Zajic. 1982ba. Hydrogen-gas Production with Citrobacter intermedius and Clostridium pasteurianum. J. Chem. Tech. Biotechnol. 32:496-502.
Iyer, P., M. A. Bruns, H. Zhang, S. Van Ginkel, and B. E. Logan. 2004. Hydrogen gas production in a continuous flow bioreactor using heat-treated soil inocula. Appl. Microbiol. Biotechnol. 89(1):119-127.
Kalia, V. C., et al. 1994. Fermentation of biowaste to H2 by Bacillus licheniformis. World Journal of Microbiol & Biotechnol. 10:224-227.
Kosaric, N. and R. P. Lyng. 1988. Chapter 5: Microbial Production of Hydrogen. In Biotechnology, Vol. 6B. editors Rehm & Reed. pp 101-137. Weinheim: Vett.
Logan, B. E., S. -E. Oh, I. S. Kim, and S. Van Ginkel. 2002. Biological hydrogen production measured in batch anaerobic respirometers. Environ. Sci. Technol. 36(11):2530-2535.
Logan, B. E. 2004. Biologically extracting energy from wastewater: biohydrogen production and microbial fuel cells. Environ. Sci. Technol., 38(9):160A-167A
Madigan, M. T., J. M. Martinko, and J. Parker. 1997. Brock Biology of Microorganisms, Eighth Edition, Prentice Hall, N.J.
Nandi. R. and S. Sengupta. 1998. Microbial Production of Hydrogen: An Overview. Critical Reviews in Microbiology, 24(1):61-84.
Noike et al. 2002. Inhibition of hydrogen fermentation of organic wastes by lactic acid bacteria. International Journal of Hydrogen Energy. 27:1367-1372
Oh, S. -E., S. Van Ginkel, and B. E. Logan. 2003. The relative effectiveness of pH control and heat treatment for enhancing biohydrogen gas production. Environ. Sci. Technol., 37(22):5186-5190.
Prabha et al. 2003. H₂-Producing bacterial communities from a heat-treated soil Inoculum. Appl. Microbiol. Biotechnol. 66:166-173
Wang et al. 2003. Hydrogen Production from Wastewater Sludge Using a Clostridium Strain. J. Env. Sci. Health. Vol. A38(9):1867-1875
Yokoi et al. 2002. Microbial production of hydrogen from starch-manufacturing wastes. Biomass & Bioenergy; Vol. 22 (5):389-396.

What is claimed is:

1. A method for identifying microorganisms suitable for biodegradation or bioremediation, comprising the steps of:
selecting a waste material,
heating the waste material to an increased temperature,
introducing the waste material into a bioreactor,
forming microorganism-containing biofilm on one or a multiplicity of substrates, wherein the substrates are coated with a gelatinous matrix for baiting nonparaffinophilic microorganisms,
selecting nonparaffinophilic microorganisms from the biofilm as nonparaffinophilic microorganisms able to biodegrade the waste material, and
isolating the nonparaffinophilic microorganisms.

2. The method of claim 1, wherein the gelatinous matrix is formed from agar and at least one carbon compound.

3. The method of claim 2, wherein the carbon compound is selected from the list consisting of glucose, fructose, glycerol, mannitol, asparagines, casein adonitol, l-arabinose, cellobiose, dextrose, dulcitol, d-galactose, inositol, lactose, levulose, maltose, d-mannose, melibiose, raffinose, rhamnose, sucrose, salicin, d-sorbitol, d-xylose or combination thereof.

4. The method of claim 1, further comprising the step of adjusting the pH of the waste material in the bioreactor to a pH between about 3.5 to 6.0 pH at any point during the method.

5. The method of claim 1, wherein the one or a multiplicity of substrates are affixed to the bioreactor, the interior portion accessible from outside the bioreactor through one or a multiplicity of openings in the bioreactor.

6. The method of claim 1, wherein the substrates are selected from the list consisting of pipes, rods, beads, slats, tubes, slides, screens, honeycombs, spheres, objects with latticework, or objects with holes or passages bored through the surface.

7. The method of claim 1, wherein the bioreactor further includes an alginate coating.

8. The method of claim 1, further comprising the step adding concentrated amounts of the isolated nonparaffinophilic microorganisms to additional waste material to biodegrade the waste material.

9. The method of claim 1, wherein the waste material is heated to a temperature of about 60 to 100° C.

10. The method of claim 1, wherein the waste material is provided by collecting the waste material in a reservoir directly from an industrial process, wherein the waste material is an effluent from the industrial process.

11. A method for identifying microorganisms suitable for biodegradation or bioremediation, comprising the steps of:
selecting a waste material,
heating the waste material to an increased temperature,
introducing the waste material into a bioreactor,
forming microorganism-containing biofilm on one or a multiplicity of substrates,
selecting nonparaffinophilic microorganisms from the biofilm as nonparaffinophilic microorganisms able to biodegrade the waste material, and
isolating the nonparaffinophilic microorganisms.

12. The method of claim 11, further comprising the step of monitoring the pH levels of the waste material at any point of the method.

13. The method of claim 11, further comprising the step of adjusting the pH of the waste material between about 3.5-6.0 pH at any point of the method.

14. The method of claim 11, wherein the one or a multiplicity of substrates are affixed to the bioreactor, the interior portion accessible from outside the bioreactor through one or a multiplicity of openings in the bioreactor.

15. The method of claim 11, wherein the substrates are selected from the list consisting of pipes, rods, beads, slats, tubes, slides, screens, honeycombs, spheres, objects with latticework, or objects with holes or passages bored through the surface.

16. The method of claim 11, further comprising the step adding concentrated amounts of the isolated nonparaffinophilic microorganisms to additional waste material to biodegrade the waste material.

17. The method of claim 11, wherein the waste material is heated to a temperature of about 60 to 100° C.

18. The method of claim 11, wherein the waste material is provided by collecting the waste material in a reservoir directly from an industrial process, wherein the waste material is an effluent from the industrial process.
